(19) United States
(12) Patent Application Publication
(10) Pub. No.: US 2026/0144983 A1
(43) Pub. Date: May 28, 2026

(54) PATIENT SPECIFIC ELECTRODE ARRAY CONDITIONING TO REDUCE INSERTION FORCES

(71) Applicant: MED-EL Elektromedizinische Geräte GmbH, Innsbruck (AT)

(72) Inventors: Max FRÖLICH, Hannover (DE); Daniel SCHURZIG, Hannover (DE); Sarah VORMELCHER, Hannover (DE); Timo BRÄCKER, Celle (DE); Onhintz DE OLANO DIETERICH, Hannover (DE); Thomas LENARZ, Hannover (DE)

(73) Assignee: MED-EL Elektromedizinische Geräte GmbH, Innsbruck (AT)

(21) Appl. No.: 19/150,084

(22) PCT Filed: Jan. 22, 2024
(86) PCT No.: PCT/US2024/012420

§ 371 Date: Jul. 22, 2025

Related U.S. Application Data
(60) Provisional application No. 63/481,125, filed on Jan. 23, 2023.

(51) Int. Cl. A61N 1/05; G06F 30/10; G06T 7/00; G06T 7/55; G06T 12/30

(52) U.S. Cl. CPC A61N 1/0541; G06F 30/10; G06T 7/0012; G06T 7/55; G06T 12/30; G06T 2207/30008; G06T 2207/30196; G06T 2210/41

(57) ABSTRACT

A patient-specific method of conditioning a cochlear electrode array so as to reduce insertion forces is provided. The method includes obtaining a 3D phantom of at least a portion of a scala tympani of a patient. The cochlear electrode array is inserted into and removed out of the 3D model of the scala tympani one or more times, to condition (e.g. soften) the electrode prior to insertion into the patient's scala tympani. Trauma when inserting the electrode into the cochlea is thus reduced.

### CROSS-REFERENCE TO RELATED APPLICATIONS

This application claims priority to U.S. Provisional Patent Application 63/481,125, filed Jan. 23, 2023, which is hereby incorporated herein by reference in its entirety.

### TECHNICAL FIELD

The present invention relates to a system and method of cochlear implant electrode insertion, and more particularly to reducing the trauma to the cochlea when inserting the electrode.

### BACKGROUND ART

A normal ear transmits sounds as shown in FIG. 1 through the outer ear 101 to the tympanic membrane (eardrum) 102, which moves the bones of the middle ear 103, which in turn vibrate the oval window and round window openings of the cochlea 104. The cochlea 104, a cross-section of which is shown in more detail in FIG. 2, is a long narrow duct wound spirally about its axis for approximately two and a half turns. The cochlea 104 includes an upper channel known as the scala vestibuli 206 and a lower channel known as the scala tympani 208, which are connected by the cochlear ductus 210. The scala tympani 208 forms an upright spiraling cone with a center called the modiolus 212 where the spiral ganglion cells of the acoustic nerve 113 reside. In response to received sounds transmitted by the middle ear 103, the fluid filled cochlea 104 functions as a transducer to generate electric pulses that are transmitted to the cochlear nerve 113, and ultimately to the brain. Hearing is impaired when there are problems in the ability to transduce external sounds into meaningful action potentials along the neural substrate of the cochlea 104.

In some cases, hearing impairment can be addressed by an auditory prosthesis system such as a cochlear implant that electrically stimulates auditory nerve tissue with small currents delivered by multiple stimulation contacts distributed along an implant electrode. FIG. 1 shows some components of a typical cochlear implant system where an external microphone provides an audio signal input to an external signal processing stage 111 which implements one of various known signal processing schemes. The processed signal is converted by the external signal processing stage 111 into a digital data format, such as a sequence of data frames, for transmission into a receiver processor in an implant housing 108. Besides extracting the audio information, the receiver processor in the implant housing 108 may perform additional signal processing such as error correction, pulse formation, etc., and produces a stimulation pattern (based on the extracted audio information) that is sent through an electrode that is implanted into the cochlea 104. The electrode may include an electrode lead 109 and an electrode array 110, which at least partially penetrates into the cochlea 104 through a surgical opening in the outer surface of the cochlea 104. Typically, this electrode is an array 110 that includes multiple stimulation contacts 112 on its surface that deliver the stimulation signals to adjacent neural tissue of the cochlea 104 which the brain of the patient interprets as sound. The individual stimulation contacts 112 may be activated sequentially or simultaneously in one or more contact groups.

The insertion of the electrode into the cochlea 104 and through the scala tympani 208, is not trivial, but rather a major surgery that involves full anesthesia and usually takes from 1.5 to 5 hours. There is a large variance between individual cochlea sizes and shapes. If an electrode would be sized and/or pre-conditioned in accordance with a “one fits all” model, the bending radius of the electrode within the cochlea 104 might not fit to each and every individual. Especially in small cochleae the conditioning effect would not have been utilized fully. Furthermore, if the sizing/pre-conditioning radius in the apical part of the electrode is smaller than a minimum threshold, the risk of electrode tip fold-over rises due to the apical part of the electrode being bent too far while inside the larger diameter basal part of the cochlea.

Trauma can occur when inserting the electrode into the cochlea. The electrode array 110 penetrates into the cochlea 104 through a surgical opening called a cochleostomy and enters into the scala tympani 208. The electrode array 110 has multiple electrode contacts on or slightly recessed below its outer surface for applying one or more electrical stimulation signals to target audio neural tissue within the cochlea 104. The extra-cochlear electrode lead 109 that goes from the implant housing 108 to the cochleostomy opening usually has no electrical contacts except perhaps a ground electrode and it encloses connecting wires that deliver electrical stimulation signals to the electrode contacts on the electrode array 110. Insertion of the electrode array 110 into the cochlea 104 can cause trauma to the cochlear tissue due to the rigidity, friction, and impact of moving the electrode array 110 through the cochlea 104. For example, insertion of the electrode array 110 may damage soft tissues, membranes, thin bony shelves, blood vessels, neural elements, etc. In the case of multiple insertions, the damage can accumulate.

### SUMMARY OF THE EMBODIMENTS

In accordance with an embodiment of the invention, a patient-specific method of conditioning a cochlear electrode array so as to reduce insertion forces is provided. The method includes obtaining a 3D phantom of a scala tympani of a patient. The cochlear electrode array is inserted into and removed out of the 3D phantom of the scala tympani one or more times, to condition (e.g. soften) the electrode prior to insertion into the patient's scala tympani. Trauma when inserting the electrode into the cochlea is thus reduced.

In accordance with related embodiments of the invention, obtaining the 3D phantom may include obtaining an image of a cochlea of a patient. Shape information of the cochlea is derived from the image. A 3D model of at least a portion of the scala tympani based on the shape information of the cochlea is created. The method further includes performing one of 1) fabricating the 3D phantom based on the 3D model of the scala tympani, or 2) selecting the 3D phantom from a set of pre-fabricated 3D phantoms which anatomically best matches the 3D model.

In accordance with further related embodiments of the invention, the 3D phantom may be fabricated based on the 3D model of at least a portion of the scala tympani. Creating the 3D model of the scala tympani may include fitting a model of a cochlea cross-section from previous subject(s) to the derived shape of the cochlea. The shape information of the cochlea may be a shape of the cochlea lateral wall and/or dimensions of the cochlea lateral wall.

In accordance with still further related embodiments of the invention, the method may further include inserting the electrode array into the scala tympani of the patient. Obtaining the 3D phantom may include rapid prototyping and/or sterilizing the 3D phantom. Obtaining the image of the cochlea may include computerized tomography (CT). The method may further include sterilizing the electrode array, followed by packaging the sterilized electrode array.

In accordance with yet further related embodiments of the invention, a kit for use by any of above-described embodiments is provided. The kit includes the electrode array and a set of pre-fabricated 3D phantoms.

In accordance with related embodiments of the invention, the electrode array and/or the set of pre-fabricated 3D phantoms in the kit may be sterilized. The set of pre-fabricated 3D phantoms may include at least a 3D phantom for a small cochlea and a 3D phantom for a large cochlea, wherein the size of the cochlea is measured according to the A value, B value and/or the H value.

### BRIEF DESCRIPTION OF THE DRAWINGS

The foregoing features of embodiments will be more readily understood by reference to the following detailed description, taken with reference to the accompanying drawings, in which:

FIG. 1 shows a conventional cochlear prosthesis;

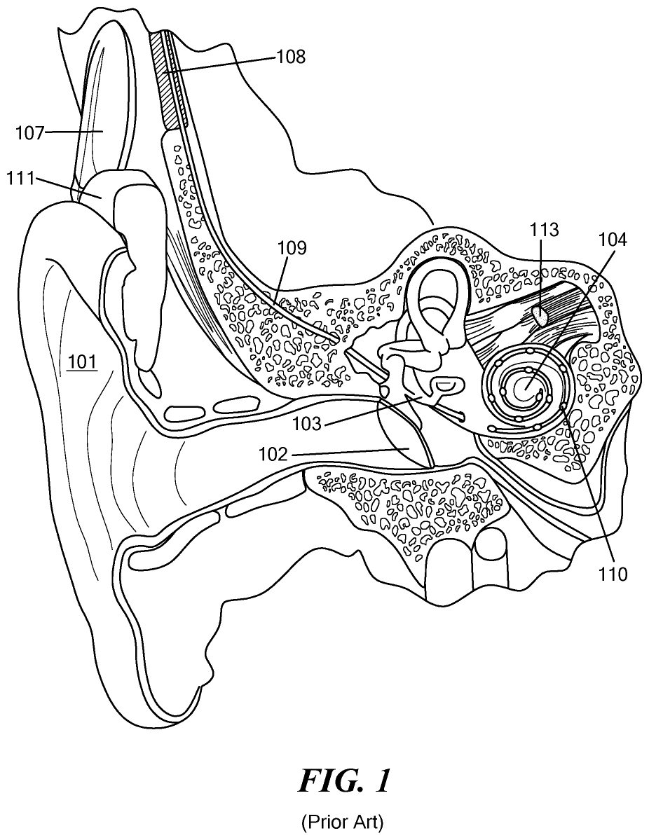

FIG. 2 shows a cross-section of a cochlea;

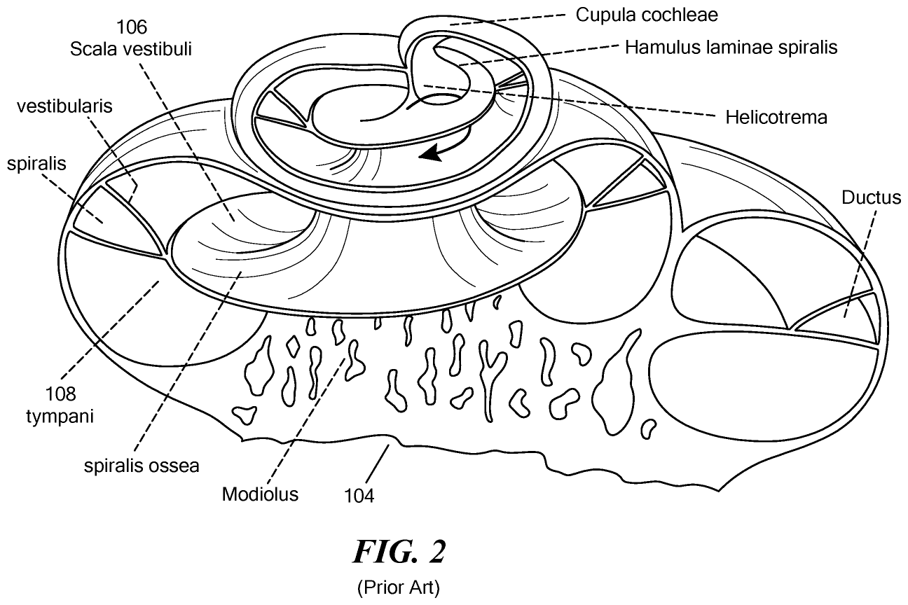

FIG. 3 is a process flow diagram of an exemplary patient specific method of conditioning a cochlear implant electrode array to reduce insertion forces, in accordance with an embodiment of the invention.

FIG. 4 is a process flow diagram of an exemplary method of fabricating a phantom of a patient's scala tympani, in accordance with an embodiment of the invention.

FIGS. 5(a) and 5(b) shows various assessment of the cochlear lateral wall using clinical CT imaging, in accordance with an embodiment of the invention. FIG. 5(c) shows cross-sections view of a cochlea microanatomy model, in accordance with an embodiment of the invention. FIG. 5(d) shows an isotropic view of a cochlea microanatomy model, in accordance with an embodiment of the invention. FIG. 5(e) shows a patient specific 3D phantom of a scala tympani of a patient, in accordance with an embodiment of the invention.

### DETAILED DESCRIPTION OF SPECIFIC EMBODIMENTS

In illustrative embodiments, a patient-specific method of conditioning a cochlear electrode array to reduce insertion forces is provided. The method includes obtaining a 3D phantom of a scala tympani of a patient. The cochlear electrode array is inserted into and removed out of the phantom one or more times, to condition (e.g., soften) the electrode prior to insertion into the patient's scala tympani. Trauma when inserting the electrode into the cochlea is thus reduced. Details are described below.

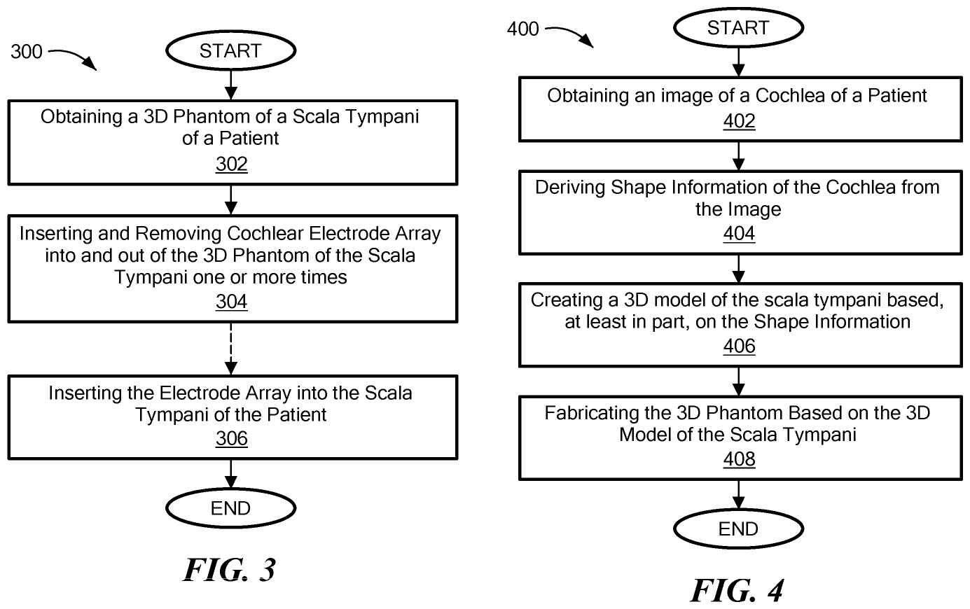

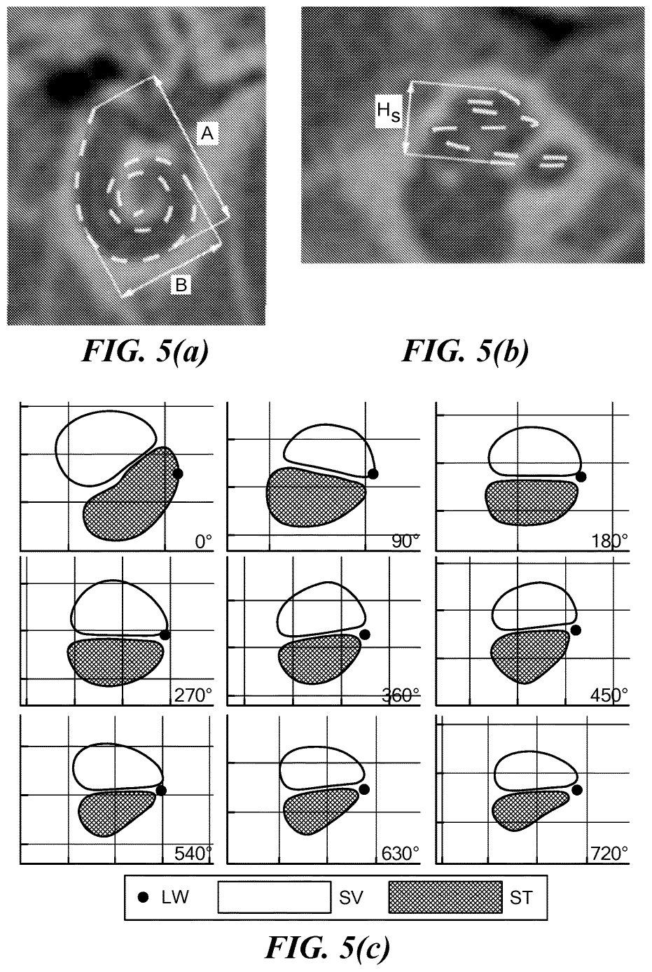

FIG. 3 is a process flow diagram 300 of an exemplary patient specific method of conditioning a cochlear implant electrode array to reduce insertion forces, in accordance with an embodiment of the invention. At stage 302, the method includes obtaining a 3D phantom of a scala tympani of a patient. As used in this description and the accompanying claims, the term “phantom” shall mean, unless the context otherwise requires: a physical model of the body; or of a specific part or portion of a specific part thereof. The entire scala tympani may not be included in the physical model. Illustratively, the 3D phantom may be a 3D model of the scala tympani, except that it does not include the outer wall of the scala tympani. The 3D phantom obtained in Stage 302 is patient specific in that it is physical model of all, or at least a portion of that particular patient's scala tympani, with the dimensioning thereof. Further details regarding the fabrication of such a phantom is described below with regard to FIG. 4.

At stage 304, the method further includes inserting and removing the cochlear electrode array into and out of the 3D phantom of the scala tympani one or more times. In this manner, patient specific pre-conditioning of the electrode array (i.e., prior to insertion of the electrode array into the cochlea of the patient) is achieved. The amount of strain softening depends on the stress the electrode is exposed to during this pre-conditioning phase. Consequently if bent in a smaller radius, the effect will be more pronounced. There is a large variance between individual cochlea sizes and shapes. If the electrode is pre conditioned in a “one fits all” model, the bending radius might not fit to each and every individual. Especially in small cochleae, the conditioning effect would not have been fully realized.

The first insertion creates different insertion force profiles than all subsequent insertions. The first profile often shows greater overall insertion forces and/or more peaks. After an electrode array has been inserted several times, forces are reduced and the force profile itself becomes smoother. This process is known for filled rubbers and described as strain softening. The softening of the electrode array may be attributed to rupture of cross links in the silicone rubber matrix of the electrode array, and also breakage between weak physical bonds between silicone rubber and platinum wire in the electrode.

After the electrode array has been preconditioned, as described above, the electrode array may be sterilized and properly packaged. In various embodiments, the 3D phantom may be sterilized after manufacturing prior to electrode insertion, such that further electrode sterilization is not necessary. During surgery, at stage 306, the electrode array is inserted into the scala tympani of the patient, advantageously with the expectation of less trauma due to the preconditioning.

FIG. 4 is a process flow diagram 400 of an exemplary method of fabricating a phantom of a patient's Scalia Tympani, in accordance with an embodiment of the invention. At stage 402, the method includes obtaining an image of a cochlea of a patient. This may often involve, computerized tomography (CT), however other imaging techniques may be used, such as an MRI scan, an optical scan, ultrasound imaging or combination thereof. In clinical CT scans, only the bony structure of the cochlea is visible and no soft tissue. Thus, to create a patient specific model of a patient's scala tympani, several steps may be needed.

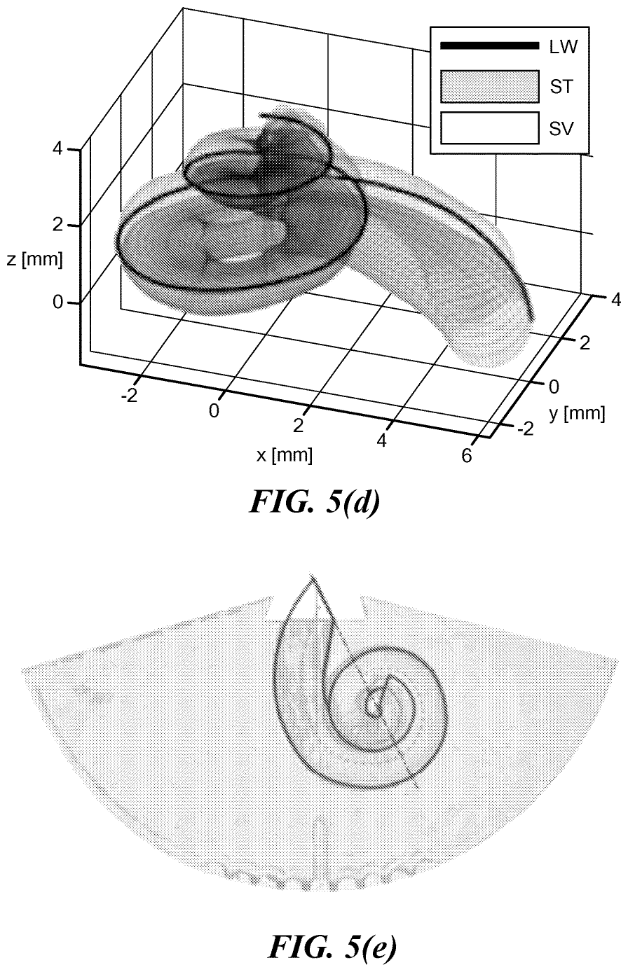

At stage 406, the method includes creating a 3D model of the scala tympani, based at least in part, of the derived shape information of the cochlea. Illustratively, this may be accomplished by referencing a database of cochlear microanatomy models. FIGS. 5(c) and 5(d) show cross-sections of the cochlear microanatomy, including the scala tympani, which may be derived, without limitation, from high resolution images of a number of cadavic cochleae. See, for example: Schurzig D, Timm M E, Majdani O, Lenarz T, Rau T S. The Use of Clinically Measurable Cochlear Parameters in Cochlear Implant Surgery as Indicators for Size, Shape, and Orientation of the Scala Tympani. Ear and hearing. July 2021; 42(4):1034-1041; and Schurzig D, Fröhlich M, Raggl S, Scheper V, Lenarz T, Rau T S. Uncoiling the Human Cochlea—Physical Scala Tympani Models to Study Pharmacokinetics Inside the Inner Ear. Life. 2021; 5:373, each of which is hereby incorporated herein by reference in its entirety. These cross-sections may then be combined with the derived shape information of the cochlea (see stage 406) to create a full volumetric representation, i.e., a 3D model, of the scala tympani (see FIG. 5(e)). For example, by fitting the cochlear microanatomy models that include the scala tympani to the derived shape information of the cochlea, the shape of the tissue of the individual can be recreated. The 3D model of the scala tympani may be oriented along its insertion axis and an insertion phantom can be created (FIG. 5(e)). Furthermore, an idealized opening of the scala tympani may be created.

At stage 408, the 3D phantom may be fabricated based on the 3D model of the scala tympani. In various embodiments, the fabrication of 3D phantom may include rapid prototyping or milling. The model of the scala tympani may be oriented along its insertion axis and an insertion phantom can be created (Fig. (e)). Furthermore, an idealized opening of the scala tympani may be created.

Alternatively, at stage 408, the 3D phantom may be selected from a set of pre-fabricated 3D phantoms. The 3D phantom anatomically best matching the measured 3D model may be selected. Calculating the anatomically best matching 3D phantom may be done by, without limitation, applying the method of least squares to the parameters of the measured 3D model and the known parameters of the pre-fabricated 3D phantoms.

The set of pre-fabricated 3D phantoms, along with the electrode array, may be provided in a kit. The electrode array and/or the set of pre-fabricated 3D phantoms in the kit may be sterilized. The set of pre-fabricated 3D phantoms may include at least a 3D phantom for a small cochlea and a 3D phantom for a large cochlea. The size of the cochlea may be measured according to the A value, B value, H value or a combination thereof.

The preconditioning is extremely easy to perform. Surgery time will only be increased by the time it takes for conditioning (<5 min). Design and manufacturing of the phantom can take place as soon as imaging data is available prior to surgery, if pre-fabricated phantoms are not provided.

The embodiments of the invention described above are intended to be merely exemplary; numerous variations and modifications will be apparent to those skilled in the art. All such variations and modifications are intended to be within the scope of the present invention as defined in any appended claims.

1. A patient-specific method of conditioning a cochlear electrode array so as to reduce insertion forces, the cochlear electrode array initially include the method comprising:
obtaining a 3D phantom of a scala tympani of a patient; and
inserting the cochlear electrode array into, and removing the cochlear electrode array out of, the 3D phantom of the scala tympani one or more times.

2. The method according to claim 1, wherein obtaining a 3D phantom includes:
obtaining an image of a cochlea of a patient;
deriving shape information of the cochlea from the image; and
creating a 3D model of at least a portion of the scala tympani based, at least in part, on the shape information of the cochlea; and
performing one of multiple alternative actions, the multiple alternative actions including 1) fabricating the 3D phantom based on the 3D model of the scala tympani, and 2) selecting the 3D phantom from a set of pre-fabricated 3D phantoms which anatomically best matches the 3D model.

3. The method according to claim 2, wherein selecting the 3D phantom includes applying a method of least squares to parameters of the 3D model and parameters of the pre-fabricated 3D phantoms.

4. The method according to claim 2, wherein the shape information is a shape of the cochlea lateral wall and/or dimensions of the cochlea lateral wall.

5. The method according to claim 2, wherein creating the 3D model of the scala tympani includes fitting a model of a cochlea cross section from previous subject(s) to the derived shape information of the cochlea.

6. The method according to claim 2, wherein obtaining the image of the cochlea includes Computed Tomography (CT).

7. The method according to claim 1, further comprising inserting the electrode array into the scala tympani of the patient.

8. The method according to claim 1, wherein obtaining the 3D phantom includes rapid prototyping.

9. The method according to claim 1, further comprising sterilizing the electrode array.

10. The method according to claim 9, further comprising packaging the sterilized electrode array.

11. A kit for use by any of the methods in claims 1, the kit including the electrode array and a set of pre-fabricated 3D phantoms.

12. The kit according to claim 11, wherein the electrode array and/or the set of pre-fabricated 3D phantoms in the kit are sterilized.

13. A system according claim 11, wherein the set of pre-fabricated 3D phantom include at least a 3D phantom for a small cochlea and a 3D phantom for a large cochlea, wherein the size of the cochlea is measured according to the A value, B value, H value or a combination thereof.
